(19) United States
(12) Patent Application Publication
(10) Pub. No.: US 2012/0203164 A1
(43) Pub. Date: Aug. 9, 2012

(54) DEVICE AND METHOD FOR DRUG DELIVERY TO A TARGETED SKIN LAYER

(76) Inventors: Gabriel Bitton, Jerusalem (IL); Ron Nagar, Tel Aviv (IL); Benny Pesach, Rosh-Ha'ayin (IL)

(21) Appl. No.: 13/499,704

(22) PCT Filed: Oct. 4, 2010
(86) PCT No.: PCT/IB10/54476

§ 371 Date: Apr. 2, 2012

Related U.S. Application Data
(60) Provisional application No. 61/247,952, filed on Oct. 2, 2009.

(51) Int. Cl. A61M 37/00

(52) U.S. Cl. 604/22; 604/506; 604/113

(57) ABSTRACT

The present invention relates to a device and method for the controlled delivery of a drug to a targeted layer of the skin in a human or animal subject. In particular, the present invention relates to such a device, system and method for selective drug delivery to the cutaneous or subcutaneous layer of skin to enhances the effectiveness of the drug delivery.

### FIELD OF THE INVENTION

The present invention relates to a device and method for the controlled delivery of a drug to a targeted layer of the skin in a human or animal subject. In particular, the present invention relates to such a device, system and method for selective drug delivery to the cutaneous or subcutaneous layer of skin to enhances the effectiveness of the drug delivery.

### BACKGROUND OF THE INVENTION

Pen injectors are useful when drug delivery by way of injection is required on a regular bases and in particular when the person delivering the drug does note have formal medical training. Such delivery devices are becoming increasingly common amongst those having chronic conditions such as diabetes where self-treatment enables such persons effectively manage their condition.

A non limiting example of usage of such devices is the area of diabetes. Diabetes is a very serious illness affecting millions of people today. In order to survive many diabetic patients require insulin injections to maintain proper blood glucose levels. Such injections of insulin require drug injection systems.

Many medical treatment systems and methods involve drug injection, systems that employ subcutaneous injections of therapeutic fluids, drugs, proteins, and other compounds. Such delivery systems and methods, especially for insulin delivery, may use injection pens to inject insulin to the subcutaneous tissue.

Current insulin injection pens are generally configured to include a disposable insulin reservoir and a disposable needle through which insulin is delivered to the tissue. In most such systems the needle is disposable intended for single dosage, while the insulin reservoir may be used for a plurality of dosages that may span two or three days.

In many instances, the subject requires that the insulin levels be tightly and continuously controlled therefore insulin delivery by way of injection may be required around the clock to ensure proper blood glucose levels.

With such delivery system primarily two types of insulin drugs may be injected. The first is a long acting insulin providing for the basal insulin needed for keeping patient's blood glucose in the desired range between to meals and over night. A second is a rapid acting insulin bolus injection that provides an amount of insulin to offset the rise in blood glucose levels resultant of the ingested foodstuff, for example, carbohydrates.

Many conventional subcutaneous injection devices are incapable of quickly matching or preventing the rise of blood glucose. The delay in such matching is also true in case of the “rapid-acting” insulin. Some of the reasons for this delay include a lag in the absorption of insulin from the injection site and the time it takes for complex insulin molecules to break down into monomers.

Additionally, since blood glucose levels rise shortly following the meal, the delay in matching insulin to the rising levels causes post prandial hyperglycemic events (i.e., when levels of blood glucose are above normal) to occur. Further, occasionally after a certain period of time passes (e.g., 2-3 hours) after a meal, the blood glucose levels drop yet insulin concentrations in the blood rise followed by the peak of the systemic insulin effect and may result in causing hypoglycemic events (i.e., when levels of blood glucose are below normal) to occur. Both hyperglycemic and hypoglycemic events are highly undesirable.

Additionally, since local blood perfusion at the insulin injection region has large variability, depending on the ambient temperature and other parameters, it induces large variations to the delay of the peak of time profile of the insulin action. Those variations in the insulin peak action period further increase the variability in the blood glucose level.

### SUMMARY OF THE INVENTION

The present invention overcomes these deficiencies of the background by providing a device and method for improved delivery of a drug, for example insulin, by targeting cutaneous and subcutaneous areas of the skin with high blood perfusion so as to improve drug absorption and pharmaceutical profile of the drug. The device and method of the present invention are particularly adapted for drug delivery with a needle based drug delivery devices or catheter based infusion sets, for example a syringe or injection pen, such that the needle depth of penetration is controllable and is oriented parallel to the skin surface allowing one to reach the targeted skin area.
An optional embodiment of the present invention provides for a method for drug delivery with a needle based drug delivery device comprising: determining a drug dose to be delivered; and activating at least one treatment element to optimize absorption of drug delivery at an injection site about the skin surface of a subject; and determining a required needle depth of penetration based on the activation of at least one treatment element; and deploying a needle to penetrate the skin surface parallel to the skin surface at the injection site and at the required depth of penetration to deliver the dose.
   An optional embodiment of the present invention provides for a method for drug delivery with a needle based drug delivery device comprising: determining a drug dose to be delivered; and deploying a needle to penetrate the skin surface, parallel to the skin surface at an injection site and at a required depth of penetration to deliver the dose.
   Optionally the depth of penetration is determined based on the location of capillary rich area.
   Optionally the parallel injection is obtained by skin surface manipulation including lifting, raising, suctioning, pulling, pinching, vacuuming or creating a skin fold at the injection site to allow for parallel needle penetration.
   Optionally the dose is delivered at a single location or at multiple locations along the drug delivery route and limited to a depth of penetration.
   Optionally and preferably the depth to which the drug may be deposited drug is from about 1 mm to about 3 mm below the skin surface.
   Optionally the drug is delivered into multiple location by injecting the drug to multiple depths at the time a needle is advanced into the tissue, and at different depths along the drug injection path.
   Optionally the drug is delivered into multiple locations is provided by a needle comprising a plurality of delivery pores along the needle length.
   Optionally the drug is delivered into multiple locations utilizing high pressure spray to penetrate the skin barrier and into the tissue under the skin barrier.
   An optional embodiment of the present invention provides for a method for drug delivery where the drug is delivered into multiple locations using micro needle array wherein a treatment is applied to the drug injection site to increase local blood perfusion.
   Optionally and preferably treatments are provided to increase local blood perfusion at the injection site.
   Optionally the delivered drug is insulin Viaject.
   An optional embodiment of the present invention provides for a method for a catheter based drug delivery system wherein drug delivery is preformed in parallel to the skin surface, the method comprising:
   inserting the catheter parallel to the skin surface at a depth from about 1 mm to about 3 mm below the skin surface; and determining a drug dose to be delivered; and delivering the dose through the delivery catheter.
   Optionally the drug is delivered in multiple locations by using multiple exit port catheter.
   An optional embodiment of the present invention provides for a method for catheter based drug delivery system wherein the catheter includes several exit ports enabling drug infusion into multiple locations, the method comprising: determining a drug dose to be delivered; and delivering the dose through the delivery catheter.
   An optional embodiment of the present invention provides for a needle based drug delivery device for injecting the drug in parallel to a skin surface at an injection site, the device comprising an injection pen or a syringe coupled with at least one skin manipulating element to enable parallel injection.
   Optionally the skin manipulating element provide for manipulating the skin at the injection site are selected from at least one or more of the group consisting of: lifting, raising, suctioning, pulling, pinching, vacuuming or any combination thereof.
   Optionally the skin manipulating element provides for creating a skin fold at the injection site to allow for parallel needle penetration.
   Optionally the treatment element comprises at least two arms that are manipulated relative to one another to pinch an area of skin.
   Optionally a heating treatment is applied with a heating element to the skin injection site.
   An optional embodiment of the present invention provides for a method for drug delivery where the treatment is applied to the drug delivery site to increase local blood perfusion and wherein the treatment is heating and heating is applied by multiple heating elements having different temperatures between 38-42 degrees Celsius and wherein the heating element further away from the drug delivery point has higher temperature to ensure high blood perfusion while keeping the temperature at the drug delivery site below 37 degrees Celsius.
   An optional embodiment of the present invention provides for a method for drug delivery where the treatment is applied to the drug delivery site and the treatment is heating and heating is switched on and then off for periods of time wherein local blood perfusion is still high from an earlier heat application.
   An optional embodiment of the present invention provides for a method for drug delivery where a treatment to increase local blood perfusion is applied to the drug delivery site and injected drug is the Viaject insulin.
   An optional embodiment of the present invention provides for a heating element for coupling and use with a drug delivery system over an injection site comprising at least two or more heating elements arranged in a concentric ring fashion forming an inner heating element and an outer heating element, and wherein the outer heating element is adapted to heat to a higher level than the inner heating element, therein forming a heat gradient; and wherein the inner heating element is disposed at or substantially at the drug delivery site.

Although the foregoing description of the device and method of the present invention is provided with examples and description relating to diabetes and use of insulin, it is not to be understood that the embodiments of the present invention are limited to use with insulin or for diabetes as a skilled artisan would appreciate that the device and method of the present invention may be extended to other disease states and/or drug both in an acute or chronic forms.

Unless otherwise defined the various embodiment of the present invention may be provided to an end user in a plurality of formats, platforms, and may be outputted to at least one of a computer readable memory, a computer display device, a printout, a computer on a network or a user.

Unless otherwise defined, all technical and scientific terms used herein have the same meaning as commonly understood by one of ordinary skill in the art to which this invention belongs. The materials, methods, and examples provided herein are illustrative only and not intended to be limiting. Implementation of the method and system of the present invention involves performing or completing certain selected tasks or steps manually, automatically, or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustrative diagram of the skin surface and its underlying layers epidermis, dermis, and hypodermis

FIG. 2A-2B show the method of injection during delivery, perpendicular injection as shown in FIG. 2A and parallel injection as show in FIG. 2B.

FIG. 3 show a schematic diagram depicting an optional method of injection of insulin in a direction parallel to the skin surface

FIG. 4A-D provide a schematic illustrative diagram of a syringe according to an optional embodiment of the present invention;

FIG. 5A-B provide a schematic illustrative diagram of a syringe according to an optional embodiment of the present invention;

FIG. 6 provides a schematic illustrative diagram of a multiport needle or catheter utilized with the device according to an optional embodiment of the present invention.

FIG. 7A-C are schematic flowcharts according to optional method of the present invention for parallel drug delivery.

FIG. 8 is a schematic illustration of a heating treatment element that may be utilized with the device and system according to the present invention.

FIG. 9 is a graphical illustration showing the improved drug delivery profile provided with a device system and method according to optional embodiments of the present invention.

### DESCRIPTION OF THE PREFERRED EMBODIMENTS

The principles and operation of the present invention may be better understood with reference to the drawings and the accompanying description. The following reference labels listed below are used throughout the drawings to refer to objects having similar function, meaning, role, or objective.

10 skin

12 needle based drug delivery device;

14 needle or catheter exit pore

100 drug dosage selector or controller

110 injector—a mechanism to inject predetermined amount of insulin;

120 needle activator—a mechanism to push the needle into the skin at a predetermined time;

130 drug reservoir;

140 needle or catheter;

150 parallel drug delivery skin manipulating element;

200 concentric heater treatment element;

205 innermost ring;

210 intermediate ring;

220 outmost ring;

240, 250 Wires, cables.

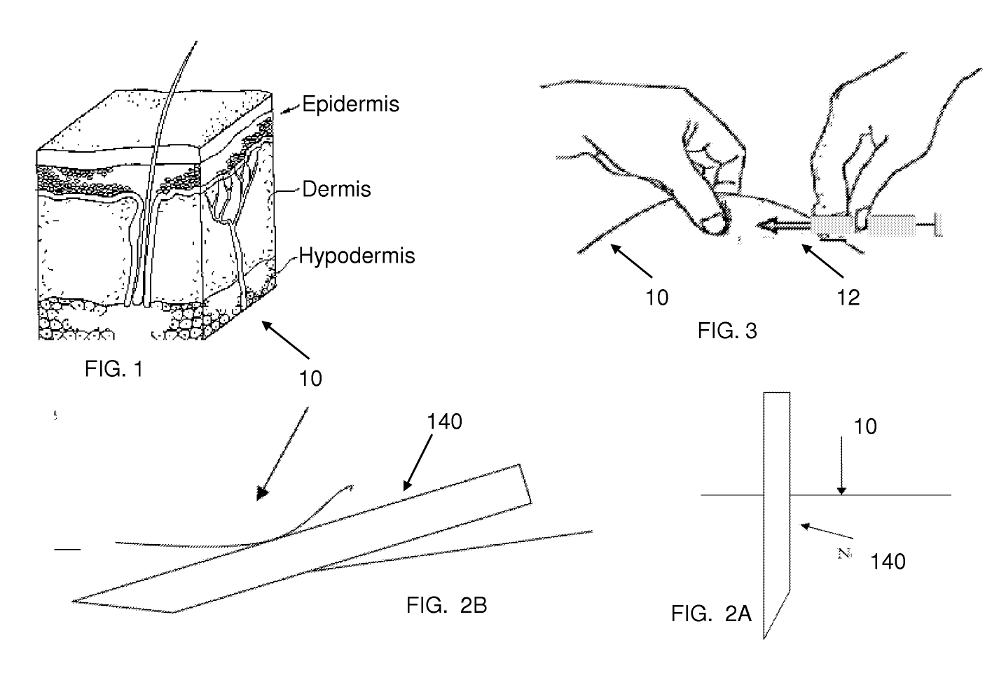

FIG. 1 shows a sectional view of skin 10 showing the different layers epidermis, dermis and hypodermis, where the dermis is primarily targeted by the device and method of the present invention due to the available blood supply within the layer, for example from about 1 mm to about 3 mm below skin surface 10.

FIGS. 2A-2B show the method of injection during delivery, perpendicular injection as shown in FIG. 2A and parallel injection as show in FIG. 2B. As indicated above studies have shown that the depth of injection to the target site which is determined by the type of delivery chosen. For example a perpendicular delivery as shown in FIG. 2A provides an injection site that is 5-9 mm below skin surface. For example a parallel delivery as shown in FIG. 2B provides an injection site that is about 1-3 mm below skin surface. Injecting using the parallel injection as shown in FIG. 2B will most preferably result in faster absorption of the substance into the blood system as the area of 1-3 mm below skin surface is more readily perfuse with blood.

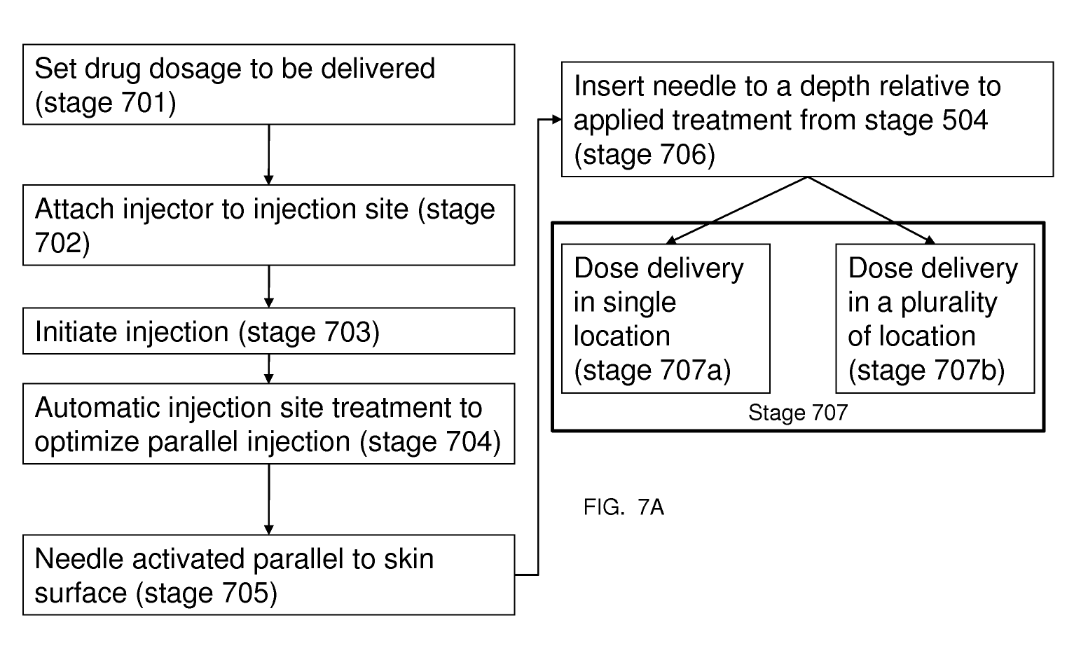

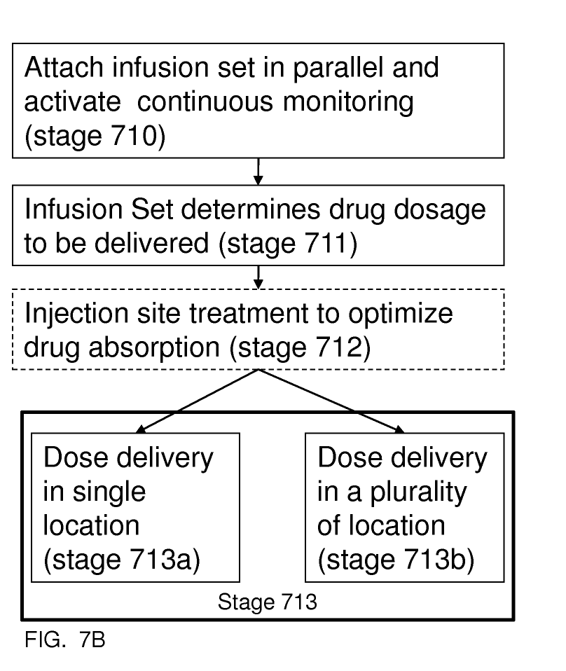

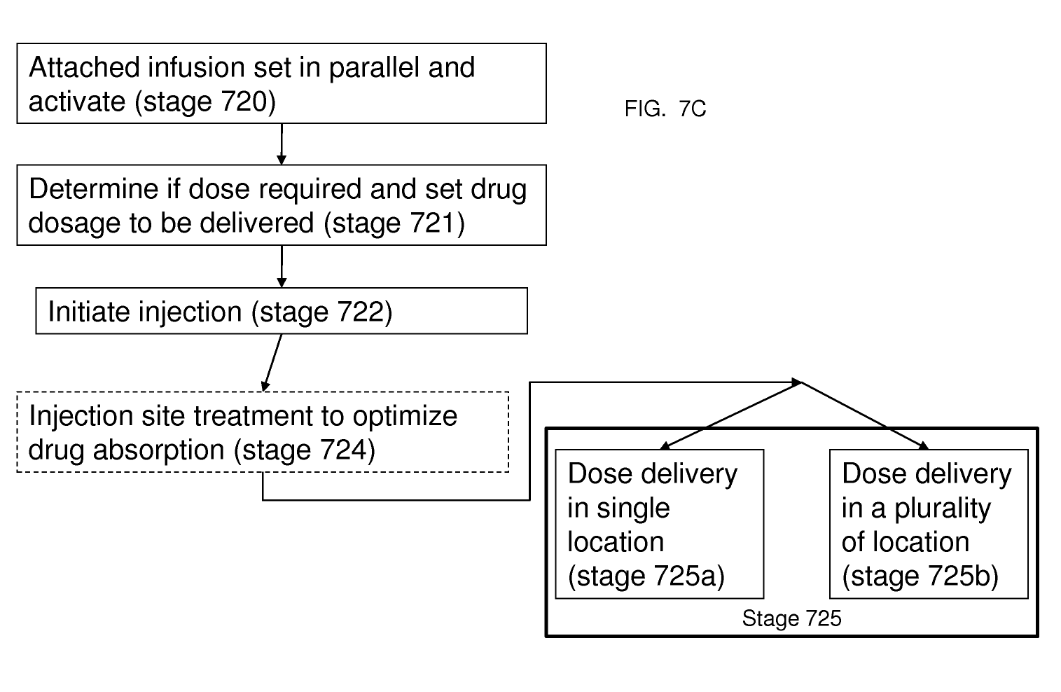

FIG. 3 depicts a person administering a drug and/or medicament by injection by pinching an area of skin 10 and injecting with injection device 12, for example a syringe or injection pen where the injection is administered parallel to the skin surface 10 as described in FIG. 2B and detailed in FIG. 7A.

Optionally the same method of parallel administration may be utilized for placing an automatic infusion set with drug delivery catheter for administering a drug and/or medicament. For example, an infusion set may be placed by pinching an area of skin 10 and then placing catheter 18 in place to allow for parallel drug delivery.

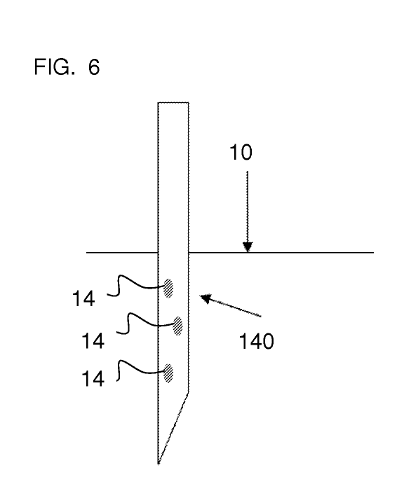

Optionally a delivery catheter may be provided with a plurality of pores 14 (FIG. 6) to allow for the delivery of the drug dosage to a plurality of locations along its length within the delivery path.

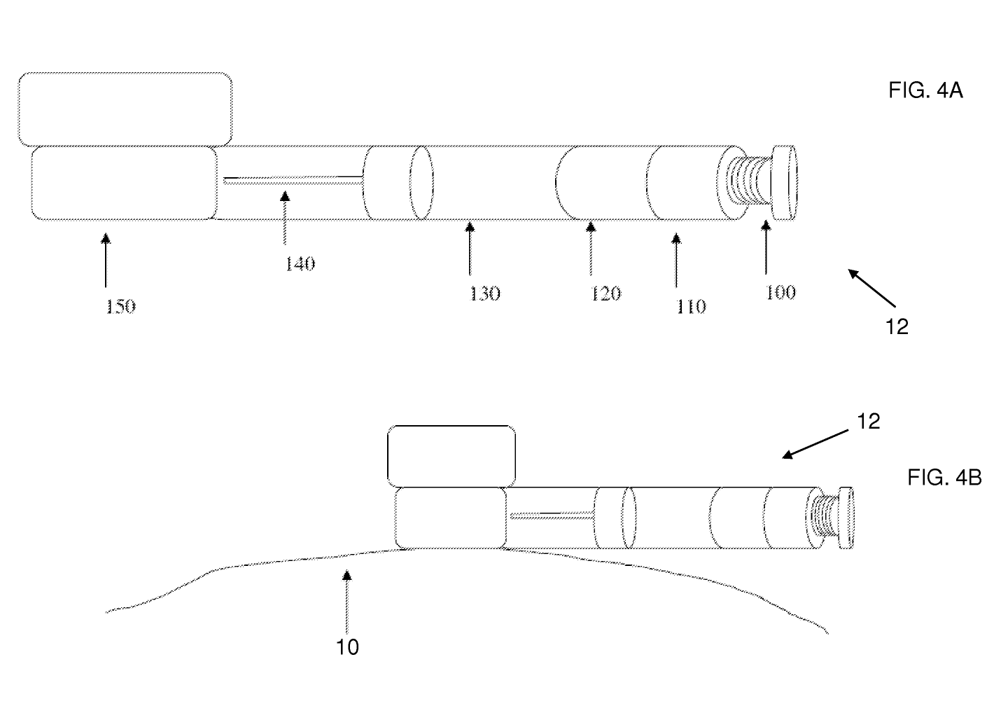

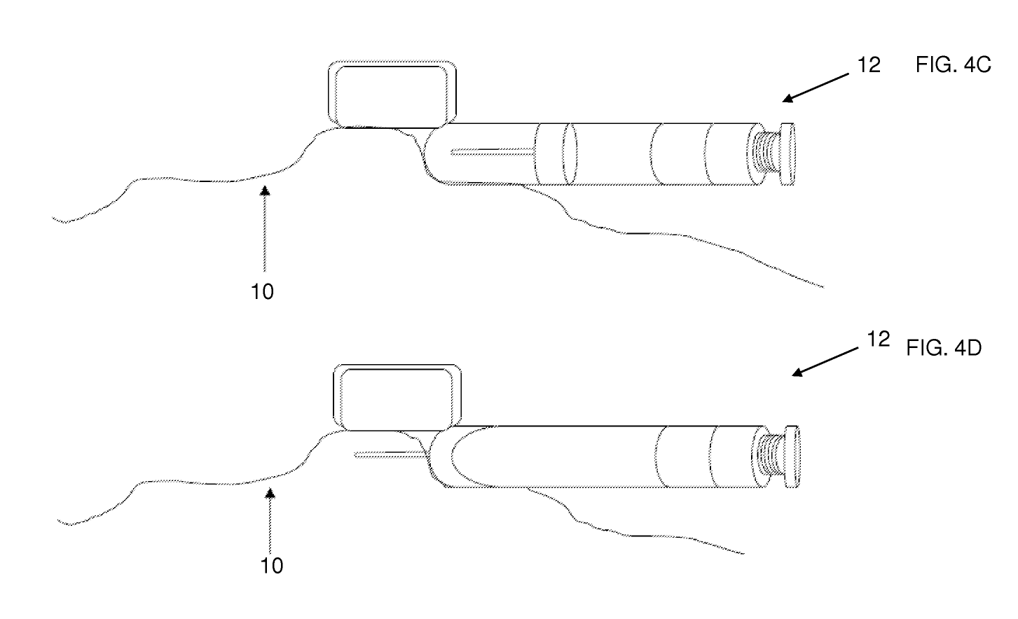

FIG. 4A-D provide a schematic illustration of a needle based drug delivery device 12 for example an injection pen or syringe, according to an optional embodiment of the present invention comprising a needle based drug delivery device 12 and a parallel delivery skin manipulating element 150 for manipulating the skin area 10 where the drug and/or medicament is to be delivered in parallel to the skin area 10 for example by lifting, raising, suctioning, pulling, pinching, vacuuming, or the like manipulation of the skin 10 most preferably creating a fold at the injection area to allow for parallel injection as shown in FIG. 2B and FIG. 3.

Optionally parallel delivery skin manipulating element 150 may be attached to and/or fit and/or integrated and/or otherwise securely coupled with the needle based drug delivery device 12.

Optionally a plurality of optional parallel delivery treatment elements 150 may be interchangeably fit and/or utilized with needle based drug delivery device 12.

Optionally and preferably needle based drug delivery device 12 comprises a dosage selector 100, injector 110, needle activator 120, drug and/or medicament reservoir 130 and needle 140.

Dosage selector 100 most preferably may be provided to select and/or control and/or otherwise adjusting the amount of drug and/or medicament, for example insulin, that is to be injected and delivered through skin surface 10 in a parallel fashion. Optionally dosage selector 100 may be provided in the form of a syringe plunger, or a control dial of an injection pen, or the like device for controlling the amount of drug to be delivered.

Injector 110 most preferably provides a mechanism to controllably inject a predetermined amount of a drug, for example insulin, as defined with dosage selector 100. Optionally injector 110 may be provided in optional forms for example including but not limited to spring based trigger, mechanical based trigger, electronic based trigger, motor based trigger, any combination thereof or the like. Optionally injector 110 may provide for multistage delivery of the dosage selected with dosage selector 100. For example, a dose of 1 ml may be delivered in a single dose or a plurality of sub-dosages while delivering the full 1 ml dosage.

Optionally injector 110 may be manually or remotely controlled. Optionally remote control of injector 110 may for example be provided by wired, wireless, cellular, RF, short range communication, RFID or the like communication device and/or protocol.

Optionally injector 110 may be controlled by interfacing and/or otherwise communicating with an auxiliary device for example including but not limited to a mobile communication device, mobile telephone, computer, PDA, timing device or the like auxiliary device.

Most preferably needle activator 120 may be provided to push and/or otherwise insert needle 140 in a parallel fashion, parallel to skin surface 10 in a controllable manner such that delivery parameters for example including but not limited to timing and/or needle distance may most preferably be controlled.

Most preferably drug and/or medicament stores 130 is provided to store a sufficient amount of drug that is to be delivered with needle 140.

An optional parallel delivery skin manipulating element 150, depicted in FIG. 4A-D, depicts a mechanism able to pull the skin away from the body to a predetermined location, for example via suctioning and/or vacuum application, therein most preferably lifting the injection area by a controllable amount creating a skin fold.

Most preferably skin manipulating element 150 communicates with injector 110 and needle activator 120 to control the depth of needle penetration in the injection area as well as to control the timing of needle 140 activation.

Optionally, parallel delivery skin manipulating element 150 may be further comprise heating element to further improve the absorption of the drug being delivered, for example insulin.

FIG. 4B shows an optional schematic depiction of a needle based drug delivery device 12 including a suctioning parallel delivery skin manipulating element 150 that is disposed over and coupled with an injection area over skin 10. FIG. 4C depicts that needle based drug delivery device 12 during the activation of parallel skin manipulating element 150 wherein the skin is lifted to a controllable level, creating a fold. FIG. 4D shows the parallel entry of needle 140 parallel to skin surface 10, where most preferably injector 110 and activator 120 control the timing of needle deployment and the depth of needle deployment so as to optimize absorption of the drug delivery.

Optionally skin 10 is lifted to a predetermined height by parallel delivery element 150 by means of adhesive tape which may be utilized to attach element 150 to the skin.

Optionally and preferably injector 110 and activator 120 provide a predetermined needle penetration depth and deployment timing based on the size of the skin fold created with parallel skin manipulating element 150.

Optionally and more preferably injector 110 and activator 120 provide dynamic control of needle penetration depth and deployment timing based on the size of the skin fold created with parallel skin manipulating element 150.

Optionally needle penetration depth may be determined to optimize the absorption and pharmaceutical profile of the drug to be delivered. For example, parallel needle based drug delivery device 12 may provide for injecting insulin or any other drug into any required shallow tissue area such as the dermal layer, the fatty layer or any required layer of the skin 10.

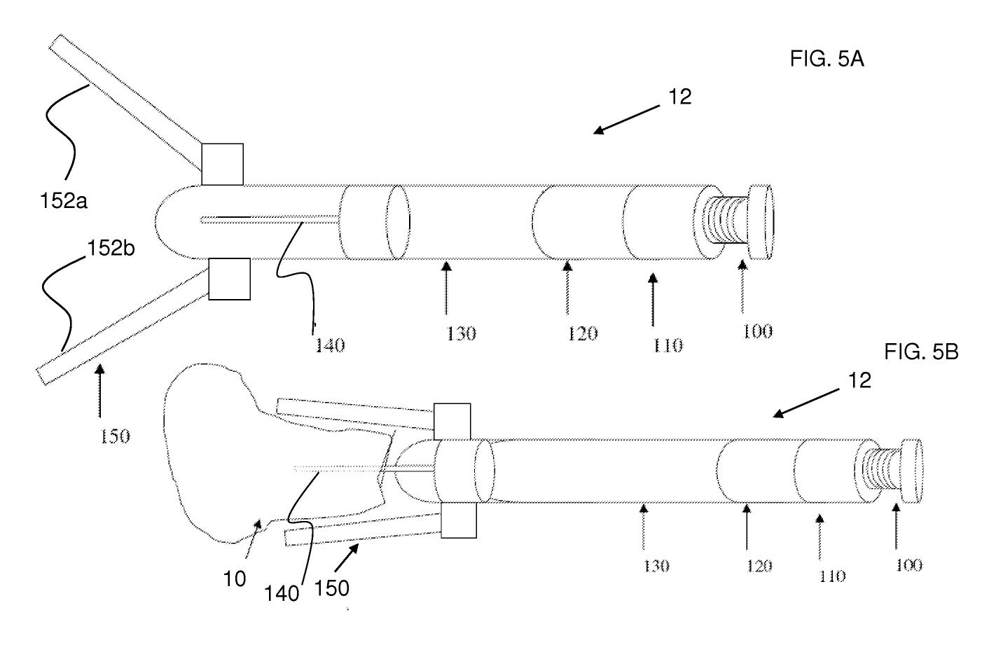

FIGS. 5A-B depict an optional embodiment of parallel needle based drug delivery device 12, as described above in relation to FIG. 4A-D, comprising a parallel drug delivery skin manipulating element 150 in the form of a mechanical pinching tool provided so as to pinch the injection area 10 prior to parallel injection.

Preferably parallel drug delivery skin manipulating element 150, of FIGS. 5A-B, in the form of a mechanical pinching tool for example may comprise at least two arms 152a, 152b that manipulate the injection area about skin 10 to pinch such that needle 140 is inserted parallel to skin surface 10. Optionally and preferably as shown in FIG. 5B needle 140 is inserted about the same plane as arms 152a, 152b providing for a parallel insertion relative to the skin surface 10.

Mechanical pinching device 150 of FIG. 5B, for example may be coupled and/or otherwise be attached most preferably parallel to the skin surface area 10, where arms 152a, 152b, are separated prior to activation. Once parallel needle based drug delivery device 12 is activated arms 152a and 152b move towards each other resulting in effectively pinching the skin surface 10 between them forming a skin fold. Optionally and preferably the size of the skin fold may be controllable and/or predetermined. Most preferably needle 140 may then advanced parallel to the skin and injects the drug in parallel to the skin at a shallow depth, such that the depth of needle 140 penetration is directly proportional and related to the size of the skin fold.

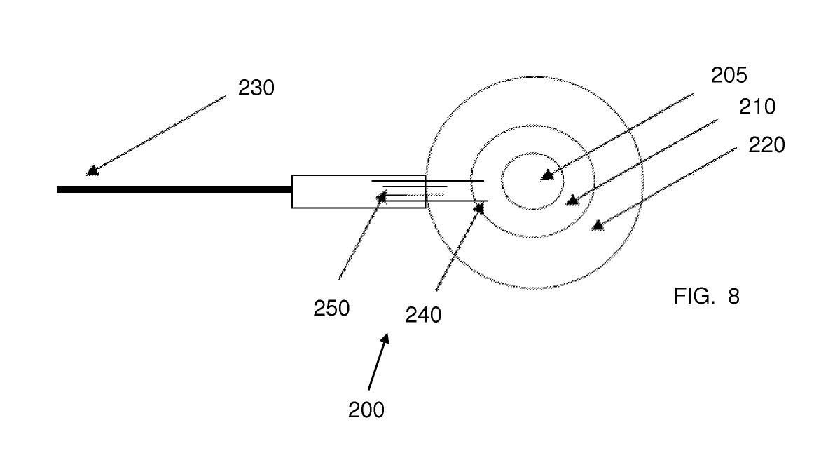

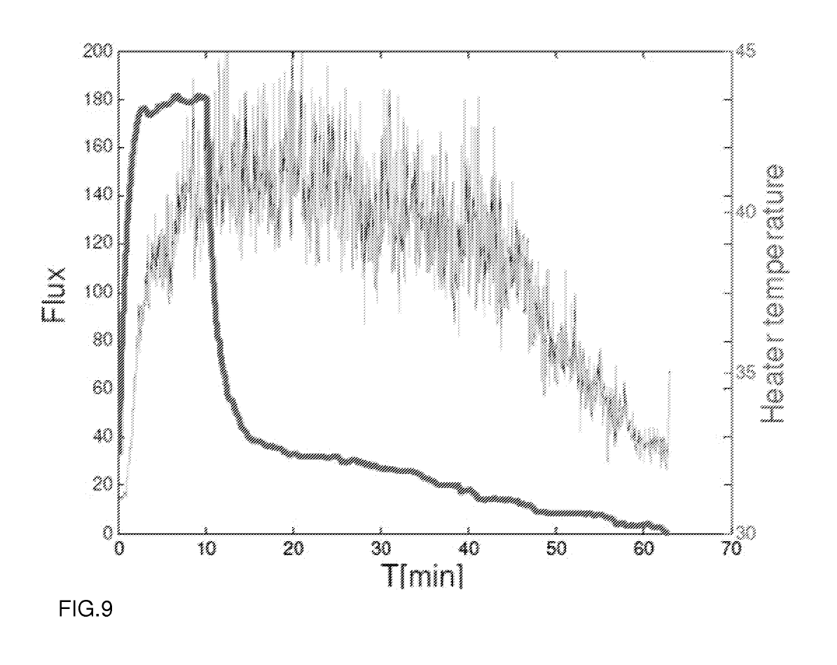

Optionally arms 152a, 152b, and/or parallel drug delivery skin manipulating element 150 may be further provided with additional treatment elements for improving or otherwise optimizing the absorption and/or pharmaceutical profile of the drug to be delivered; for example including but not limited to heating element, massage element, ultrasound element, chemical element or the like. For example, parallel drug delivery skin manipulating element 150 comprising arms 152a, 152b may further comprise a heating element, as described in FIG. 8, (or a vasodilatation chemical) may be disposed about the inner surface of at least one of arms 152a and/or 152b such that the heat is applied to the injection site skin fold 10.

Most preferably the penetration depth of needle 140 may be controlled, for example by injector 110, as previously described, wherein during the needle 140 penetration into a parallel injection site about skin surface 10 the drug dosage may be delivered in at least one or more locations and/or in a multistage manner and/or sequential manner, or trough a plurality of needle pores or the like. For example, a dose of 60 ml, needle 140 may be targeted to injected to a depth of 1.5 mm, where the dose may be delivered at three different depth locations during needle 140 penetration; for example, a first delivery at 0.8 mm 10 ml of the dose, a second delivery of 30 ml at 1.1 mm and a third delivery at 1.5 mm with the remaining 20 ml of the dose.

Optionally a needle array and/or a plurality of needles may be utilized to deliver the drug dose to a plurality of locations about skin surface 10 in a parallel manner.

Optionally the length of the needle in the last description of the pen, FIGS. 4A-D and 5A-B, may be long enough to inject the drug far enough from the entry point. Also the pen may include mechanism to inject at multiple locations and hence deposit the injected drug at multiple locations. This will most preferably further increase the absorption of the drug into the blood and it will also reduce the local amount of blood so as to not cause pain due to extra fluid that is deposited locally. It is known that injection of even small amount of fluid into the shallow skin layers can cause temporary expansion of the skin and a feeling of pain. By injecting the drug to multiple locations the pain may be alleviated.

Most preferably parallel injection procedure and device according to optional embodiments of the present invention may be used also to inject a drug into the dermal layer without the drawbacks that were described above that are generally associated with injection into a precise depth into the dermal layer. For example in Mantoux tuberculin test a purified protein derivative is injected into at a shallow angle into the skin surface using a thin needle. The accuracy of this process is important in order not to have false negative test results. Most preferably utilizing the injector described above may allow one to accurately inject the drug into the target tissue with precise and easy method.

The rate of drug absorption to the blood system depends also on the local concentration of the drug, the higher the concentration the slower the absorption. Hence injection of a larger amount of drug will be slowly absorbed compared to the absorption of a smaller amount of drug as it would require a larger amount of interstitial body fluid to dilute it. To overcome this problem optional embodiments of the device and method of the present invention provide for injecting the drug to multiple locations rather than a single location. In some embodiments the drug is injected to different depth by using a mechanism that advances the needle from one depth to another depth while injecting some of the dose to the different depths. This may be done while the needle travels in or out from the skin. Optionally and preferably advancing mechanism may comprise a spring to push the needle with different stops in which another spring may be used to push a certain portion of the drug. Alternatively an electric motor may be used to advance the needle to certain depths and to push pre-determined portion of the drug to the different depths. Optionally the device according to optional embodiment of the present invention may be suited to be embedded in a pen type of injection but could also be used with a syringe. In this case the syringe is inserted into a cylinder that will optionally and preferably push the whole syringe to the predetermined depth and push the syringe cap to deposit a pre-determined amount of drug at pre-determined depths. This way the local amount of the drug will be smaller and could be absorbed faster. Distribution the drug into a larger volume will increase the rate of drug absorption as the drug is diluted by a larger amount of body fluid.

Optionally needle 140 may be provided with a plurality of pores 14 as shown in FIG. 6, for delivery along a single path at different locations. Preferably needle 140 of FIG. 6 is adept at distributing the drug to a plurality of different location substantially simultaneously, optionally and preferably reducing the local concentration of the injected drug in an attempt to increase drug absorption and subject discomfort.

FIG. 7A depicts a flowchart of a method for parallel drug delivery according to the present invention and as described with respect to the optional embodiments of FIGS. 4A-D and 5A-B respectively. In stage 701 a drug dose is set by a subject, optionally a dosage may be set by a healthcare giver, or determined and set automatically for example by an automatic monitoring system (FIGS. 7B-C). Once the dose is set, in stage 702 a needle based drug delivery device, for example an injection pen or syringe, is attached or otherwise coupled to the injection site about skin surface 10.

Next in stage 703 injection is initiated, for example to apply a local treatment about the injection site for example including but not limited to activating a heating element, applying a chemical, applying a vasodilating agent, massage, piezoelectric element, ultrasound or the like treatment element to enhance drug delivery.

Next in stage 704 a parallel drug delivery skin manipulating element 150 may be activated for example to pinch a skin area about the injection site, as described in FIGS. 4A-D or FIGS. 5A-B.

Next in stage 705 needle penetration is activated most preferably parallel to the skin 10 surface at the injection site. Optionally and preferably needle penetration depth is determined so as to optimize drug absorption and pharmaceutical profile. Most preferably needle penetration depth is determined based on the activation and results of the parallel drug delivery skin manipulating element 150, of stage 704.

Next in stage 706 needle penetration is initiated in a parallel manner to the depth determined in stage 705. Finally in stage 707 dosage delivery is provided optionally to a single location stage 707a at the determined depth, or optionally the drug dose set in stage 701 is delivered in a controllable manner about a plurality of injection locations in stage 707b optionally each injection site may be individually with controllable in terms of local dose and depth.

FIG. 7B depicts a flowchart of a method for parallel drug delivery according to the present invention utilizing an automatic infusion. The method initiates in stage 710 where an infusion set is coupled to a subject and initiate in automatically monitoring and controlling drug delivery, for example by checking glucose levels and insulin doses to maintain glucose homeostasis. Next in stage 711 the automatic infusion set determined the dosage required.

Optionally in stage 712 optional injection treatment elements for example including but not limited to heating, massaging, local administration of vasodialating chemicals or the like.

Next once the dose is set, in stage 713 the drug is delivered through at least one or more delivery catheters. Optionally the dose may be delivered in one location, stage 713a, or multiple locations in stage through a plurality of pores 14.

FIG. 7C depicts a flowchart of a method for parallel drug delivery according to the present invention utilizing an infusion set for example. The method initiates in stage 720 where an infusion set is coupled to a subject and initiated for monitoring and controlling drug delivery, for example by periodically or according to a schedule, checking glucose levels. Next in stage 721 the dose required is determined by infusion set and the infusion set is programmed to delivery the required dose through at least one or more delivery catheters. Next in stage 722 drug delivery is initiated with the infusion set.

Optionally next in stage 724 optional injection treatment elements for example including but not limited to heating, massaging, local administration of vasodialating chemicals or the like.

Next once the dose is set and the injection area primed, stage 725 he drug is delivered through at least one or more delivery catheters,. Optionally the dose may be delivered in one location, stage 725a or multiple locations in stage 725b through a plurality of pores 14.

FIG. 8 shows a heating element configuration, that may optionally be integrated or otherwise coupled with needle or catheter based drug delivery device 12 or catheter based drug delivery device, for example an injection pen and/or syringe or drug infusion catheter; or integrated with a parallel drug delivery skin manipulating element 150. FIG. 8 shows heating element 200 comprising a plurality of concentric ring heating elements 205, 210, 220 that may be disposed around the injection or infusion site 205 defining the center where optionally and preferably innermost ring 205 is not actively heated while it may passively heat. Optionally a temperature gradient about rings 205, 210 and 220 may be applied such that the highest temperature is in the outmost ring 220 and the lowest temperature is the innermost ring 205.

Optionally inner ring 205 may be heated to a temperature that will not damage the drug, the further away rings 220 will heat to a higher temperatures to increase local blood perfusion but still not damage the drug. It is known that the effect of local heat on blood perfusion extends several centimeters away from the heating site. FIG. 8 shows such a heater 200 comprising multiple single heaters which may be more effective in improving blood perfusion compared to a single heater ring. The inner space 205 is the area where the drug is injected and heat is not applied to. The second area 210 is the first heater which is set to a temperature that will not increase the injected drug above a certain threshold which will damage the drug. Area 220 is the second heater which is farther away from the drug point of injection and it is set for a higher temperature which will increase local blood perfusion further while still keeping the temperature at the drug injection site below the threshold temperature. Wires 240 and 250 preferably form the wires connecting the inner heater and outer heater respectively to the power source and controller. Cable 250 is a bundle of all electrical cables extending from the heater. Note that the numbers of rings may be two or more and the shape of the heater may be rings shape, U shaped, oval shaped. Another embodiment of such a heater is a heater where the heating element shape could be square or ellipse or circle and the injection site is on the side of theater. And the heating device has several heating elements which may heat to different temperatures, the heating element which is closest to the injection site is heated to a temperature of about 38.5 degrees Celsius, a heating element which is farther away is heated to a higher temperature and in the same manner a heating element which is further may be heated to a higher temperature n this way optimization of the heating process is achieved. The induced blood perfusion is high while the temperature at the injection site is kept below a temperature which may damage the injected drug.

Optionally and preferably the injected drug is insulin. Optionally and preferably the drug is not injected but infused to the tissue using an infusion catheter.

US patent application US2007000821230, incorporated herein by reference as if fully set forth, describes methods and apparatus for increasing local blood perfusion which improves the delivery into the blood system of a drug injected into the subcutaneous tissue, incorporated herein by reference. The methods described in this patent may be further improved by using modified spatial heating profile. By increasing the heated area around the injection site the effect on insulin absorption into the blood may be increased. For example, the effect of the device on insulin PK was tested by comparing insulin concentration in the blood with and without the device in a meal tolerance test protocol for two different heating pads with different heating areas. It was found that increasing the heating area from 8.2 cm² to 17.5 cm² increases the effect of the InsuPatch device on PK profile. The small heating element improved the area under the curve of insulin delivery during the first hour (AUC1 hr) by 30% while the larger heating element improved the AUC1 hr by 45%.

US patent application US2007000821230, herein incorporated by reference as if fully set forth, describes methods and apparatus for increasing local blood perfusion which improves the delivery into the blood system of a drug injected into the subcutaneous tissue. The methods described in this patent may be further improved by using modified temporal heating profile. It is known from the literature that heating the tissue increases local blood perfusion and that this effect stays on even if the heating is switched off for some time.

Using such a heating profile may save power needed to increase blood perfusion. It is known that Laser Doppler (‘LDF’) signal is a marker for cutaneous blood perfusion and may be used to track changes in blood perfusion FIG. 8 shows laser Doppler signal as function of time when the tissue was heated for 10 minutes and then switched off. The heater used in this experiment is a ring type heater with inner diameter of 1 cm and outer diameter of 2.5 cm. The heater was placed on the skin. The LDF signal was recorded from a sensor placed in the middle of the LDF ring at a location that was in the center of heating but it was not heated. The graph shows the heater temperature and the laser Doppler signal as function of time. At time T=0 the heater is switched on and the temperature rises from 32° C. to a temperature of 42° C. and the laser Doppler signal rises from about 20 units to 160 units. The heating is stopped after 10 minutes and in about 2 minutes the skin temperature goes back to the starting temperature while the Doppler signal stays at above 100 for additional 35 minutes. This result suggests that to increase blood perfusion heating does not need to be “on” for the whole period, the heating may be switched off while still maintaining high blood perfusion. The heating profile may be composed of a single heating for 10 minutes and then switched off or it may be composed of off and “on” heating intervals of few minutes for example heating may be “on” and followed by longer period of off

In another embodiment all the delivery methods described above are used with the Viaject™ sinulin which is ultrafast insulin drug was developed by Biodel™.

In another embodiment the methods for increasing blood perfusion such as heating massaging and other method listed on US patent application US2007000821230, herein incorporated by reference as if fully set forth, are applied to an insulin injection site where the insulin injected is the Viaject from Biodel. Adding the blood perfusion increasing methods such as heat or massage may help also reduce pain and irritation that may occur in some cases of Viaject insulin injection.

While the invention has been described with respect to a limited number of embodiments, it will be appreciated that many variations, modifications and other applications of the invention may be made.

1.-28. (canceled)

29. A method for subcutaneous drug delivery with a needle based or catheter based drug delivery device at a drug delivery site, the method comprising:
a. determining a drug dose to be delivered subcutaneously;
b. applying local treatment by activating a tissue treatment element to increase blood perfusion about the drug delivery site with at least one of a heating element, applying a chemical, applying a vasodilating agent, massage, piezoelectric element, ultrasound, suction, pinching or vacuuming ; and
e. activating a mechanism to controllably inject said dose in accordance with a predefined injection process, wherein said mechanism is physically coupled to an injector and wherein, during operation, said mechanism causes the injector to inject said dose.

30. The method of claim 29 adapted for delivery with a needle based drug delivery device further comprising:
c. forming a skin fold about the drug delivery site therein placing the injection site in position for subcutaneous parallel injection at a depth from about 1 mm to about 3 mm below the skin surface; and
d. deploying a needle to penetrate the skin surface, wherein said needle is deployed substantially parallel to said skin surface at an injection site and at a required depth of penetration to deliver said dose.

31. The method of claim 29 adapted for a subcutaneous catheter based drug delivery system wherein said catheter is positioned substantially parallel to the skin surface and wherein said catheter is inserted substantially parallel to the skin surface at a depth from about 1 mm to about 3 mm below said skin surface.

32. The method of claim 29 wherein said depth of penetration is determined based on a location of a capillary rich area.

33. The method of claim 30 wherein forming said skin fold is provided by manipulating the skin surfaces with at least one of manipulating means selected from lifting, raising, suctioning, pulling, pinching, vacuuming.

34. The method of claim 30 wherein said needle comprises a plurality of exit ports and wherein, during operation, said dose is delivered at multiple depths at said injection site.

35. The method of claim 29 where the drug is delivered to multiple locations along the drug delivery route by controllably advancing the needle into the injection site in accordance with said predefined injection process.

36. The method of claim 29 further comprising the step of applying a high pressure spray to cause said drug to be distributed into tissue under said skin barrier.

37. The method of claim 29 wherein said drug is insulin.

38. The method of claim 29 wherein said dose is delivered by activating at least one of mechanism selected from the group consisting of a spring based trigger, mechanical based trigger, electronic based trigger, or motor based trigger.

39. The method of claim 29 wherein said predefined injection process comprises a first injection of a first dose level, a wait period during which said needle advances within said injection site, and a second injection of a second dose level.

40. The method of claim 29 wherein said local treatment is applied to the drug delivery site to increase local blood perfusion and wherein the treatment is heating and wherein said heating is applied by a heating member having multiple concentric heating elements adapted to provide for different temperatures between 38-42 degrees Celsius about individual elements; wherein the heating element further away from the drug delivery point has higher temperature to ensure high blood perfusion while keeping the temperature at the drug delivery site below 37 degrees Celsius.

41. The method for drug delivery according to claim 40 wherein said heating is switched on and then off for periods of time, wherein local blood perfusion is still high from an earlier heat application.

42. A needle based drug delivery device for performing the method of claim 30, said device comprising:
a. an injection pen or a syringe coupled with at least one skin manipulating element for forming said skin fold to enable said substantially parallel injection;
b. a tissue treatment element provided to increase blood perfusion about the drug delivery site with at least one of a heating element, applying a chemical, applying a vasodilating agent, massage, piezoelectric element, ultrasound, suction, pinching or vacuuming.

43. The device of claim 42 wherein said skin manipulating element is selected from at least one of or more selected from the group consisting of: lifting, raising, suctioning, pulling, pinching, vacuuming or any combination thereof.

44. The device of claim 43 wherein said skin manipulating element comprises at least two arms that are manipulated relative to one another to pinch an area of skin therein forming a skin fold.

45. The device of claim 42 wherein said tissue treatment element is a heating element comprising: at least two or more heating members arranged in a concentric fashion forming an inner heating member and an outer heating member, wherein said outer heating member is adapted to heat to a higher temperature level than said inner heating member, therein forming a heat gradient; and wherein said inner heating member is substantially disposed about the drug delivery site.

46. A heating element provided as a tissue treatment element for a drug delivery device or system, the heating element centered about an injection or infusion site of said drug delivery device, the heating element comprising a plurality of concentrically disposed heating elements and configured to form a temperature gradient about said injection or infusion site.

47. The heating element of claim 46 wherein said temperature gradient is lowest about the innermost heating element and highest at the outermost heating element.

48. The heating element of claim 46 wherein the innermost heating element is heated to a temperature of about 38.5 degrees Celsius; and wherein the outermost heating element is heated to a higher temperature.
